(19) United States
(12) Patent Application Publication
(10) Pub. No.: US 2011/0086115 A1
(43) Pub. Date: Apr. 14, 2011

(54) Vegetal-Derived, Water-Absorbing Composition

(76) Inventors: Michel F. Mercier, Mountainside, NJ (US); My Trinh Luu, Orangeburg, NY (US)

(21) Appl. No.: 12/703,012

(22) Filed: Feb. 9, 2010

Related U.S. Application Data
(63) Continuation-in-part of application No. 12/595,569, filed on Oct. 12, 2009.

(51) Int. Cl. A61K 8/25; A61Q 1/04; A61Q 5/06; A61Q 5/12; A61Q 19/08

(52) U.S. Cl. 424/724

(57) ABSTRACT

A vegetal-derived, water-absorbing composition comprising (i) a vegetal cholesterol derived semi-synthetically from one or a mixture of botanical materials from Dioscorea composita, Dioscorea terpinapensis, Dioscorea villosa and/or Trillium erectum, (ii) a non-animal, botanically-derived phospholipid comprising at least 20% phosphatidylcholine, (iii) a botanical oil or ester derivative at a concentration of from 50% to 90% by weight, based on the total weight of the water-absorbing composition, (iv) at least one sucrose ester at a concentration of from 5% to 10% by weight, based on the total weight of the water-absorbing composition, and (v) fumed silica at a concentration of 8% to 15% by weight, based on the total weight of the water-absorbing composition. The vegetal-derived, water-absorbing composition is capable of absorbing between 200% and 400% of its weight of water and may be used as a substitute for lanolin.

### CROSS REFERENCE TO RELATED APPLICATIONS

This application is a continuation-in-part of co-pending U.S. patent application Ser. No. 12/595,569 filed on Oct. 12, 2009, the disclosure of which is incorporated by reference in its entirety.

### FIELD OF INVENTION

The present invention provides a vegetal-derived, water-absorbing composition, useful in the preparation of personal care products (for topical application to human skin and hair), especially emulsions for use as vehicles for topical application of skincare or haircare active ingredients and/or medicaments. The vegetal-derived, water-absorbing composition of the present invention is capable of absorbing between 200% and 400% of its weight in water.

### BACKGROUND OF INVENTION

Lanolin is extracted from sheep wool grease; it is a complex mixture of esters formed by the esterification of long-chain carboxylic acids and hydroxycarboxylic acids with high molecular weight alcohols, cholesterol and lanosterols. Lanolin is a well-known, multifunctional ingredient in topical formulations. In addition to its high water absorbing capacity, lanolin is used as an emulsifier (or co-emulsifier) and imparts skin-softness. However, lanolin has several notable limitations. Products containing lanolin have been reported to cause allergic reactions. This can be exacerbated by the presence of high levels of fragrance incorporated in formulations to mask the odor associated with lanolin. Consequently, there has been and remains a need for a lanolin substitute. In response to this need, synthetic lanolin-like materials have been developed, including those described in U.S. Pat. Nos. 4,868, 7,217,432 and 5,436,006.

More recently, in response to consumer demands for “natural” products, lanolin substitute compositions derived from vegetable materials, which are lanolin-free, have been developed. Two such examples are described in U.S. Pat. Nos. 7,217,432 and 7,220,432. The compositions of the present invention provide a natural alternative to lanolin, with added aesthetic and performance benefits. The vegetal-derived, water-absorbing compositions of the present invention have a silky, dry after feel and provide skin barrier protection and long lasting hydration. In addition to their emolliency and moisturizing properties, the compositions of the present invention provide an effective delivery system for oil-soluble active ingredients.

### SUMMARY OF THE INVENTION

The present invention is direct to a vegetal-derived, water-absorbing composition comprising (i) a vegetal cholesterol derived semi-synthetically from one or a mixture of botanical materials from Dioscorea composita, Dioscorea terpinapensis, Dioscorea villosa and/or Trillium erectum, (ii) a non-animal, botanically-derived phospholipid comprising at least 20% phosphatidylcholine, (iii) a botanical oil or ester derivative at a concentration of from 50% to 90% by weight, based on the total weight of the water-absorbing composition, (iv) at least one sucrose ester at a concentration of from 5% to 10% by weight, based on the total weight of the water-absorbing composition, and (v) fumed silica at a concentration of 8% to 15% by weight, based on the total weight of the water-absorbing composition. The vegetal-derived, water-absorbing composition of the present invention is capable of absorbing between 200% and 400% of its weight of water.

### DETAILED DESCRIPTION OF THE INVENTION

The water-absorbing composition of the present invention is described as “vegetal-derived” because all required components that are not inorganic can be obtained from natural sources, for example, plants.

As used in the present application, by the term vegetal cholesterol derived semi-synthetically from a plant of the Dioscorea species and/or Trillium erectum is meant a mono-unsaturated, secondary alcohol of a cyclopentanophenanthrene ring structure having the empirical formula C₂₇H₄₆O that is synthesized by a process having cholesterol acetate as an intermediate. The cholesterol acetate is hydrolyzed, producing the claimed vegetal cholesterol.

In one preferred embodiment of the present invention, the vegetal cholesterol is extracted and purified from a plant of the Dioscorea species.

In a further preferred embodiment, the vegetal cholesterol is extracted and purified from a mixture of D. composita, D. terpinapensis and/or D. villosa.

In another preferred embodiment, the vegetal cholesterol is extracted and purified from Trillium erectum.

In a particularly preferred embodiment, the vegetal cholesterol is extracted and purified from at least two of D. composita, D. terpinapensis, D. villosa and T. erectum.

Vegetal cholesterol derived semi-synthetically from a plant of a species of the Dioscorea genus selected from the group of D. composita, D. terpinapensis and/or D. villosa and/or from Trillium erectum is present at a concentration of at least about 0.01%, preferably at a concentration of at least about 0.05%, more preferably at a concentration of at least about 0.1% based on the total weight of the water-absorbing composition. (Unless otherwise noted, percentages are by weight of the referenced composition.)

Vegetal cholesterol produced semi-synthetically from species of Dioscorea (D. composita, D. terpinapensis and D. villosa) and/or Trillium erectum is available from MMP Inc. (South Plainsfield, N.J.) under the tradename Vegechol®.

A second component of the vegetal-derived, water-absorbing composition of the present invention is a botanically-derived phospholipid. As used in the present application, by the term phospholipid is meant a glycerol moiety on which two fatty acids are esterified at the carbon-1 and carbon-2 positions, and a phosphate group is esterified at the carbon-3 position. The fatty acid groups which are typically esterified to the glycerol may be saturated or unsaturated C₁₂-C₂₀ fatty acids, and are preferably selected from the group consisting of palmitic, stearic, oleic, linoleic, linolenic and arachidonic acids.

The phosphate group on the phosholipid may be substituted or unsubstituted. In its unsubstituted form, the phospholipid is phosphatidic acid. Substituents on the phosphate group are well-known to those of skill in the art and may include nitrogen-containing alcohols (e.g., ethanolamine, serine and choline) and inositol (hexahydroxycyclohexane).

A preferred botanically-derived phospholipid is lecithin, preferably isolated and purified primarily from soybeans, and comprises at least 20% phosphatidylcholine. Still more preferably, the soy phosphatidylcholine is comprised of at least 20% linoleic acid. Soybean-derived lecithin comprising at least 20% phosphatidylcholine is available from MMP Inc. as part of the Vegephos® complex (INCI: Zea Mays (Corn) Oil (&) Sucrose Tetrastearate Triacetate (&) Lecithin (&) Glyceryl Undecylenate (&) Cholesterol). In the vegetal-derived, water-absorbing composition of the present invention, the botanically-derived phospholipid comprises 3% to 20% of the composition. In a particularly preferred embodiment, the water-absorbing composition of the present invention comprises phosphatidylcholine at a concentration of from 0.5 to 3% by weight of the water-absorbing composition.

Botanical oils having an HLB of 6 or 7, medium polarity esters of linear fatty acids, medium polarity esters of branched fatty alcohols, silicones selected from the group of volatile silicone fluids (preferably cyclomethicones), polydimethylsiloxanes, and phenyl-substituted silicone fluids (preferably phenyl trimethicone).

Preferred botanical oils having an HLB of 6 or 7 are selected from the group consisting of: Prunus Amygdalus Dulcis (Sweet Almond) Oil; Amaranthus Hypochondriacus (Amarinth) Seed Oil; Borago Officinalis (Borage) Seed Oil; Echium Plantagineum Seed Oil; Oenothera Biennis (Evening Primrose) Oil; Hydrogenated Castor Oil; Simmondsia Chinensis (Jojoba) Seed Oil; Avena Sativa (Oat) Kernel Oil; Olea Europaea (Olive) Fruit Oil (Olive Oil); Perilla Ocymoides Seed Oil; Plukenetia Volubillis (Inca Inchi) Seed Oil; Cannabis Sativa Seed Oil (Hemp Oil); Cucurbita Pepo (Pumpkin) Seed Oil (Pumpkin Seed Oil); Punica Granatum (Pomegranate) Seed Oil; Sambucus Nigra (Elderberry) Oil; Glycine Soja (Soybean) Oil; Fragaria Annassa (Strawberry) Seed Oil; and Helianthus Annuus (Sunflower) Seed Oil.

By “medium polarity ester” is meant an ester having a polarity index of from 15 mN/m to 30 mN/m, preferably from 20 mN/m to 30 mN/m.

Preferred medium polarity esters of linear fatty acids and medium polarity esters of branched fatty alcohols are selected from the group consisting of: C₁₂₋₁₅ Alkyl Benzoate; Caprylic/Capric Triglyceride; Cetearyl Isononanoate; Cetearyl Octanoate; Cetyl Palmitate; Diisopropyl Dimer Dilinoleate; Ethylhexyl Cocoate; Ethylhexyl Palmitate; Ethylhexyl Stearate; Isodecyl Oleate; Isononyl Isononanoate; Isopropyl Isostearate; Isopropyl Myristate; Isopropyl Palmitate; Isostearyl Isostearate; Octyl Palmitate; Pentaerythrityl Tetraisostearate; Propylene Glycol Dicaprylate/Dicaprate; Triethylhexanoin; Trimethylolpropane Triisostearate; Propanediol Dicaprylate.

In certain embodiments of the present invention, the finished formulation (topically-applied emulsion) may make labeling claims that the final product meets the certification standards of governmental and/or non-governmental organizations with respect to the “natural” or “organic” content of the product. In embodiments in which an EcoCert certification is to be claimed, the following medium polarity esters of linear fatty acids and medium polarity esters of branched fatty alcohols are preferably used: Caprylic/Capric Triglyceride; Myristyl Myristate, Isopropyl Myristate; Isopropyl Palmitate; Propanediol Dicaprylate.

As used in the present application, by the term “volatile” is meant fluids having a vapor pressure of at least about 2 mm of mercury at 20° C.

The vegetal-derived, water-absorbing compositions of the present invention include at least one sucrose ester. The person skilled in the art recognizes that sucrose esters usually consist of a mixture of mono-, di-, and triesters, and that sucrose ester types vary not only based on the degree of esterification (mono-, di-, or tri-esters) but also with respect to fatty acids used in esterification (lauric, palmitic, stearic). Preferred sucrose esters useful in the practice of the present invention include sucrose tetrastearate diacetate, sucrose laurate, sucrose palmitate, sucrose stearate, sucrose distearate, and sucrose polystearate.

Fumed silica is an important component of the vegetal-derived, water-absorbing composition of the present invention. Fumed silica useful in the practice of the present invention can be made by vapor phase hydrolysis of silicon tetrachloride. The fumed silica useful in the practice of the present invention comprises agglomerates of silicon dioxide of about 325 mesh or less. Cab-O-Sil® (Cabot Corp., Boston, Mass.) is an example of a fumed silica useful in the practice of the present invention.

Fumed silica is used is an amount of about 8% to about 15% of the total weight of the vegetal-derived, water-absorbing composition.

In preferred embodiments, the vegetal-derived, water-absorbing composition of the present invention comprises an ester of undecylenic acid at a concentration of from 0.1% to 5.0%, based on the total weight of the composition. Preferably, the ester of undecylenic acid is glyceryl undecylenate, the ester of glycerin and undecylenic acid.

The water-absorbing composition of the present invention can be used in its hydrated state, or can be hydrated during the manufacturing process (i.e., preparation of emulsion). One aspect of the present invention is directed to use of the vegetal-derived, water-absorbing composition of the present invention in emulsions comprised of water phase and one or two phases that are immiscible in water—either oil or silicone. More particularly, the vegetal-derived, water-absorbing composition of the present invention may be “dual-phase” with water as the external phase (oil-in-water; silicone-in-water) or with water as the internal phase (i.e., water-in-oil; water-in-silicone). Emulsions of the present invention may also be “three-phase” systems (e.g., water-in-oil-in-water; oil-in-water-in-oil).

Emulsions comprising the vegetal-derived, water-absorbing composition of the present invention may be in the form of a cream, lotion, serum or gel. The vegetal-derived, water-absorbing composition of the present invention is typically present at a concentration of at least about 3% by weight of the total emulsion.

The International Cosmetics Dictionary and Handbook published by the Cosmetic Toiletry and describes a wide variety of non-limiting cosmetic and pharmaceutical ingredients commonly used in personal care products that are suitable for inclusion in topically-applied formulations comprising vegetal-derived, water-absorbing composition of the present invention. Among these are skin-conditioning and hair-conditioning agents (e.g., humectants, moisturizers and conditioners), as well as “active” ingredients, including antioxidants/free radical scavengers, hydroxy acids, peptides, vitamins and derivatives, sunscreens, and rheological modifying agents, as set out in U.S. Pat. No. 6,492,326, Col. 5, line 35-Col. 20, line 52, the disclosure of which is incorporated herein by reference.

In a preferred embodiment of this aspect of the present invention, the vegetal-derived, water-absorbing composition of the present invention further includes an antioxidant, which may function as a free radical scavenger.

Non-limiting examples of antioxidants/free radical scavengers which may be used in the present invention include: Scutellaria Baicalensis Root Extract; Chamomilla Recutita (Matricaria) Extract; Citrus Reticulata Peel Extract; Pueraria Lobata Root Extract; Pueraria Lobata Root Extract; Nordihydroguaiaretic Acid; ascorbic acid and its salts and derivatives as well as derivatives of ascorbyl esters of fatty acids (e.g., magnesium ascorbyl phosphate, sodium ascorbyl phosphate, ascorbyl sorbate); tocopherol, tocopherol sorbate, tocopherol acetate, other esters of tocopherol.

The following examples are further illustrative of the present invention and serve to provide those of ordinary skill in the art with a disclosure and description of how to make and use personal care products comprising the vegetal-derived, water-absorbing composition of the present invention. The examples are representative, and should not be construed to limit the scope of the invention. While efforts have been made to ensure accuracy with respect to the numerical values in the examples (e.g., amounts, temperatures, etc.), it should be taken into account that some degree of error and deviation is possible. Unless indicated otherwise, parts are parts by weight.

### Example 1

### Moisturizing Anti-Aging Cream

| Phase | Ingredient | INCI Name | % (wt/wt) |
|---|---|---|---|
| A | DI Water | Water | 73.30 |
| A | Crystalcast ® MM (MMP) | Beta-Sitosterol (&) Sucrose Stearate | 5.50 |
| | | (&) Sucrose Distearate (&) Cetyl | |
| | | Alcohol (&) Stearyl Alcohol | |
| A | Tetrasodium EDTA | Tetrasodium EDTA | 0.10 |
| A | Clearogel ® CS 11D (MMP) | Scleroglucan | 0.25 |
| A | Polycast ® 3 (MMP) | Polyglycerin-3 | 5.00 |
| B | Clearocast ® 550 (MMP) | Isododecane (&) Isononyl | 5.00 |
| | | Isononanoate | |
| | Clearocast ® 200 (MMP) | Dimethicone (&) Dimethicone/Vinyl | 5.00 |
| | | Dimethicone | |
| | Vegenolin ® (MMP) | Zea Mays (Corn) Oil (&) Silica (&) | 3.00 |
| | | Sucrose Tetastearate Triacetate (&) | |
| | | Lecithin (&) Cholesterol (&) | |
| | | Tocopherol (&) Glyceryl | |
| | | Undecylenate | |
| C | Preservative | Preservative | 0.30 |
| C | Guanidine Complex ™ PL-MM | Polyglycerin-3 (&) Butylene Glycol | 2.50 |
| | (MMP) | (&) Water (&) Aminoguanidine | |
| | | Hydrochloride (&) Pentylene Glycol | |
| | | (&) Methylpropanediol (&) Pueraria | |
| | | Lobata Root Extract | |
| C | Fragrance | Fragrance | 0.05 |
| | | Total = | 100.00 |

Add DI Water to a main vessel. Heat 80-85° C. When temperature reaches 80-85° C., add Crystalcast® MM. Mix with propeller agitation; hold at temperature for 30 minutes. Add Tetrasodium EDTA. Continue to mix. Add premix of Clearogel® CS11D and Polycast® 3. Mix for 15 minutes, then homogenize for 5 minutes. Cool to 60° C. At 60° C., combine Phase B to Phase A. Homogenize for 15 minutes. Continue cooling. At 45° C., add Phase C ingredients, one at a time. Continue mixing and cooling. Homogenize for 5 minutes at 34° C.

### Example 2

### Moisturizing Cream

| Phase | Ingredient | INCI Name | % (wt/wt) |
|---|---|---|---|
| A | DI Water | Water | 77.42 |
| A | Crystalcast ® MM | Beta-Sitosterol (&) Sucrose Stearate (&) | 4.00 |
| | (MMP) | Sucrose Distearate (&) Cetyl Alcohol (&) | |
| | | Stearyl Alcohol | |
| A | Tetrasodium EDTA | Tetrasodium EDTA | 0.05 |
| A | Vegeceramide ® | Hydroxypropyl Bispalmitamide MEA | 1.00 |
| | PC-104 (MMP) | | |
| A | Clearogel ® CS 11D | Scheroglucan | 0.20 |
| | (MMP) | | |
| A | Polycast ® 3 (MMP) | Polyglycerin-3 | 5.00 |
| B | Clearocast ® 550 | Isododecane (&) Isononyl Isononanoate | 5.00 |
| | (MMP) | | |
| B | Clearocast ® 200 | Dimethicone (&) Dimethicone/Vinyl | 5.00 |
| | (MMP) | Dimethicone | |
| B | Vegenolin ® (MMP) | Zea Mays (Corn) Oil (&) Silica (&) Sucrose | 2.00 |
| | | Tetrastearate Triacetate (&) Lecithin (&) | |
| | | Cholesterol (&) Glyceryl Undecylenate (&) | |
| | | Tocopherol | |
| C | Preservative | Preservative | 0.30 |
| C | Lavender Oil | Lavandula Angustifolia (Lavender) Oil | 0.03 |
| | | Total = | 100.00 |

Add DI Water to a main vessel. Heat to 80-85° C. At 80-85° C., add Crystalcast® MM. Mix with propeller agitation while holding at temperature for 30 minutes. Add Tetrasodium EDTA and Vegeceramide® PC-104. Add premix of Clearogel® CS 11D and Polycast® 3. Mix for 15 minutes. Homogenize for 5 minutes. Cooling to 60-65° C. In a separate vessel, combine ingredients of Phase B. Heat Phase B mixture to 60-65° C. At 60-65° C., add Phase B to Phase A. Homogenize for 15 minutes. Cool to 45-50° C. At 45-50° C., add Phase C ingredients to main vessel. Switch to sweep blade mixer. Continue mixing. At 34° C., homogenize for 5 minutes.

### Example 3

### Body Lotion

| Phase | Ingredient | INCI Name | % wt/wt |
|---|---|---|---|
| A | DI Water | Water | 69.20 |
| A | Sisterna ® L70-C | Sucrose Laurate | 5.00 |
| | (MMP/Sisterna) | | |
| A | Clearobact ® 2 | Pentylene Glycol (&) Caprylyl Glycol (&) | 1.00 |
| | (MMP) | Polyglyceryl-10 Laurate (&) | |
| | | Cocamidopropyl PG-Dimonium Chloride | |
| | | Phosphate | |
| A | Botaderm ™ PF-08 | Water (&) Kigelia Africana Fruit Extract | 2.50 |
| | (MMP) | (&) Polysorbate 20 (&) Trifolium Pratense | |
| | | (Clover) Flower Extract (&) Butylene | |
| | | Glycol (&) Humulus Lupulus (Hops) | |
| | | Extract (&) Alchemilla Vulgaris Extract (&) | |
| | | Salvia Officinalis (Sage) Leaf Extract (&) | |
| | | Pentylene Glycol (&) Caprylyl Glycol (&) | |
| | | Polyglyceryl-10 Laurate (&) | |
| | | Cocamidopropyl PG-Dimonium Chloride | |
| | | Phosphate (&) Hydrolyzed Soy Protein | |
| B | Vegelene ® MCT | Capric/Caprylic Triglyceride (&) Squalene | 12.50 |
| | (MMP) | | |
| B | Clearocast ® 550 | Isododecane (&) Isononyl Isononanoate | 7.50 |
| | (MMP) | | |
| B | Vegenolin ® (MMP) | Zea Mays (Corn) Oil (&) Silica (&) | 2.00 |
| | | Sucrose Tetrastearate Triacetate (&) | |
| | | Lecithin (&) Cholesterol (&) Tocopherol | |
| C | Carbopol Ultrez 21 | Acrylates/C10-30 Alkyl Acrylate | 0.30 |
| | (Noveon) | Crosspolymer | |
| D | Sodium Hydroxide | Sodium Hydroxide | QS pH to |
| | (15% sol'n) | | 5.5-6.5 |
| | | Total = | 100.00 |

Mix Phase B and C ingredient, then add B/C mixture into Phase A while stiring. Adjust pH of A/B/C mixture to 5.5 to 6.5 by addition of Phase D. Homogenize for 5 minutes with a high shear mixer.

### Example 4

### Protective Lotion

| Phase | Ingredient | INCI Name | % (wt/wt) |
|---|---|---|---|
| A | DI Water | Water | 61.50 |
| A | Crystalcast ® MM (MMP) | Beta-Sitosterol (&) Sucrose Stearate | 2.00 |
| | | (&) Sucrose Distearate (&) Cetyl | |
| | | Alcohol (&) Stearyl Alcohol | |
| A | Disodium EDTA | Disodium EDTA | 0.10 |
| A | Xanthan Gum | Xanthan Gum | 0.30 |
| A | Clearogel ® CS 11D (MMP) | Scleroglucan | 0.10 |
| A | Polycast ® 3 (MMP) | Polyglycerin-3 | 2.50 |
| A | Preservative | Preservative | 0.30 |
| B | Sofmetic ® LMP-ZnO (MMP) | Petrolatum (&) Zinc Oxide | 20.00 |
| B | Clearocast ® 100 (MMP) | Dimethicone | 1.20 |
| B | Clearocast ® 550 (MMP) | Isododecane (&) Isononyl | 5.00 |
| | | Isononanoate | |
| B | Vegenolin ® (MMP) | Zea Mays (Corn) Oil (&) Silica (&) | 5.00 |
| | | Sucrose Tetastearate Triacetate (&) | |
| | | Lecithin (&) Cholesterol (&) Glyceryl | |
| | | Undecylenate (&) Tocopherol | |
| C | Dry Flo-PC (National Starch) | Aluminum Starch Octenylsuccinate | 2.00 |
| | | Total = | 100.00 |

Add DI Water to a main vessel. Heat to 80-85° C. At 80-85° C., add Crystalcast® MM. Mix with propeller agitation, while holding at temperature for 30 minutes. Add Disodium EDTA and xanthan gum. Add premix of Clearogel® CS 11D and Polycast® 3. Add preservative. Mix for 15 minutes. Homogenize for 5 minutes. Start cooling to 70-75° C. In a separate vessel, combine ingredients of Phase B. Heat to 70-75° C. When main vessel reaches 70-75° C., switch to homogenizer mixing. Slowly add Phase B, at temperature, to main vessel. Homogenize for 5-10 minutes. Start cooling. When A/B mixture is 50-55° C., add Phase C to main vessel. Homogenize for 5 minutes. Resume loop mixing and cooling. Shutdown at 34° C.

### Example 5

### Hair Conditioning Cream

| Phase | Ingredient | INCI Name | % (wt/wt) |
|---|---|---|---|
| A | Water | Water | 86.00 |
| A | Crystalcast ® MM (MMP) | Beta-Sitosterol (&) Sucrose | 4.00 |
| | | Stearate (&) Sucrose | |
| | | Distearate (&) Cetyl Alcohol | |
| | | (&) Stearyl Alcohol | |
| A | Steapyrium Chloride | Steapyrium Chloride | 0.25 |
| A | Disodium EDTA | Disodium EDTA | 0.05 |
| A | Polycast ® 3 (MMP) | Polyglycerin-3 | 3.00 |
| A | Clearogel ® CS 11D (MMP) | Scleroglucan | 0.10 |
| A | Preservative | Preservative | 0.30 |
| B | Vegenolin ® (MMP) | Zea Mays (Corn) Oil (&) Silica | 5.00 |
| | | (&) Sucrose Tetastearate | |
| | | Triacetate (&) Lecithin (&) | |
| | | Cholesterol (&) Glyceryl | |
| | | Undecylenate (&) Tocopherol | |
| C | Ulline ® S Complex (MMP) | Oxidized Corn Oil (&) Oryza | 1.00 |
| | | Sativa (Rice) Bran Oil Extract | |
| | | (&) Nordihydroguaiaretic Acid | |
| C | Fragrance | Fragrance | 0.30 |
| D | Sodium Hydroxide (15% sol'n) | Sodium Hydroxide | QS pH |
| | | | 4.0 to 5.0 |
| | | Total = | 100.00 |

In a main vessel, charge DI Water. Heating to 80-85° C. At 80-85° C., add Crystalcast® MM to main vessel. Mix with propeller; hold for 30 minutes. Homogenize for one cycle. Switch to propeller mixing, while maintaining temperature at 80-85° C. Add remaining Phase A ingredients sequentially while mixing. Cooling to 65-70° C. Add Phase B ingredient. Homogenize for one cycle. Cool to 40-45° C. At 40-45° C., add Phase C ingredients sequentially, one at a time. Adjust pH to 4.0-5.0 with Phase D ingredient. Continue cooling. Homogenize for one cycle at 34° C.

### Example 6

### Hair Styling Cream

| Phase | Ingredient | INCI Name | % (wt/wt) |
|---|---|---|---|
| A | Water | Water | 82.35 |
| A | Crystalcast ® MM (MMP) | Beta-Sitosterol (&) Sucrose | 4.00 |
| | | Stearate (&) Sucrose Distearate | |
| | | (&) Cetyl Alcohol (&) Stearyl | |
| | | Alcohol | |
| A | Disodium EDTA | Disodium EDTA | 0.05 |
| A | Eastman AQ ™ 55S | Polyester-5 | 3.00 |
| | (MMP/Eastman) | | |
| A | Polycast ® 3 (MMP) | Polyglycerin-3 | 3.00 |
| A | Clearogel ® CS 11D (MMP) | Scleroglucan | 0.10 |
| A | Preservative | Preservative | 0.30 |
| B | Vegenolin ® (MMP) | Zea Mays (Corn) Oil (&) Silica | 5.00 |
| | | (&) Sucrose Tetastearate | |
| | | Triacetate (&) Lecithin (&) | |
| | | Cholesterol (&) Glyceryl | |
| | | Undecylenate (&) Tocopherol | |
| C | Kalamansi ™ PLG-4 Ext. (MMP) | Water (&) Citrus Madurensis | 1.00 |
| | | Fruit Juice (&) Polyglycerin | |
| C | Natural Saccharides Complex 6004 | Saccharide Hydrolysate (&) | 1.00 |
| | (MMP) | Saccharide Isomerate (&) | |
| | | Glycerin (&) Water | |
| C | Fragrance | Fragrance | 0.20 |
| D | Sodium Hydroxide (15% sol'n) | Sodium Hydroxide | QS pH |
| | | | 6.0 to 7.0 |
| | | Total = | 100.00 |

Follow same procedure in Example 5.

### Example 7

### Moisturizing Lip Treatment

| Inqredient | INCI Name | % (wt/wt) |
|---|---|---|
| Castor Oil | Ricinus Communis (Castor) Seed Oil | 43.00 |
| Vegeceramide ® PC-104 (MMP) | Hydroxypropyl Bispalmitamide MEA | 1.00 |
| Sisterna ® A10E-C (MMP/Sisterna) | Sucrose Tetrastearate Triacetate | 7.50 |
| White Beeswax (Strahl & Pitsch) | White Beeswax | 4.00 |
| Paraffin Wax SP173 (Strahl & Pitsch) | Paraffin | 1.50 |
| Palmeride ® H1 (MMP) | Hydrogenated Palm/Palm Kernel | 7.50 |
| | Glycerides | |
| Vegelene ® 90MM (MMP) | Squalene | 9.50 |
| Sofmetic ® LMP (MMP) | Petrolatum | 15.00 |
| G.M.U. STD (MMP) | Glyceryl Undecylenate | 1.00 |
| Vegenolin ® (MMP) | Zea Mays (Corn) Oil (&) Silica (&) | 5.00 |
| | Sucrose Tetastearate Triacetate (&) | |
| | Lecithin (&) Cholesterol (&) Glyceryl | |
| | Undecylenate (&) Tocopherol | |
| 40% D&C Red 7 CA Lake (and) | D&C Red 7 Ca Lake (&) | 1.00 |
| Palmeride H1 (Sun Chemical) | Hydrogenated Palm/Palm Kernel | |
| | Glycerides | |
| CWD 9901 (Sun Chemical) | Iron Oxide Red (&) Hydrogenated | 2.00 |
| | Palm/Palm Kernel Glycerides | |
| Ronastar Noble Sparks (EMD) | Calcium Aluminum Borosilicate (&) | 2.00 |
| | Silica (&) Titanium Dioxide | |
| | Total = | 100.00 |

Combine all ingredients and start heating to 80° C. Mix well. Cool to 60° C. Pour into mold.

1. A vegetal-derived, water-absorbing composition comprising
(i) a vegetal cholesterol derived semi-synthetically from one or a mixture of botanical materials from
(a) a species of the genus Dioscorea selected from the group consisting of D. composita, D. terpinapensis and D. villosa or
(b) Trillium erectum,
(ii) a non-animal, botanically-derived phospholipid comprising at least 20% phosphatidylcholine,
(iii) a botanical oil or ester derivative thereof, said botanical oil or ester derivative thereof being present at a concentration of from 50% to 90% by weight, based on the total weight of the water-absorbing composition,
(iv) at least one sucrose ester at a concentration of from 5% to 10% by weight, based on the total weight of the water-absorbing composition, and
(v) 8% to 15% by weight, based on the total weight of the water-absorbing composition, of fumed silica,
wherein the vegetal-derived, water-absorbing composition is capable of absorbing between 200% and 400% of its weight of water.

2. The vegetal-derived, water-absorbing composition of claim 1 wherein the vegetal cholesterol is present at a concentration of from 0.5% to 3.0%, based on the total weight of the vegetal-derived, water-absorbing composition.

3. The vegetal-derived, water-absorbing composition of claim 2 wherein the vegetal cholesterol is present at a concentration of from 0.1% to 1.0%, based on the total weight of the vegetal-derived, water-absorbing composition.

4. The vegetal-derived, water-absorbing composition of claim 1 wherein the source of the non-animal, botanically-derived phospholipid is soybean lecithin.

5. The vegetal-derived, water-absorbing composition of claim 4 wherein the soybean lecithin is present at a concentration of from 3% to 20%.

6. The vegetal-derived, water-absorbing composition of claim 5 wherein the soybean lecithin is present at a concentration of from 5% to 20%.

7. The vegetal-derived, water-absorbing composition of claim 1 wherein the at least one sucrose ester is selected from the group consisting of sucrose tetrastearate, sucrose triacetate, sucrose polystearate, sucrose laurate, and combinations thereof.

8. The vegetal-derived, water-absorbing composition of claim 1 further comprising an ester of undecylenic acid at a concentration of 0.1 to 5.0%, based on the total weight of the vegetal-derived, water-absorbing composition.

9. The vegetal-derived, water-absorbing composition of claim 8 wherein the ester of undecylenic acid is present at a concentration of from 1.0 to 2.5%, based on the total weight of the vegetal-derived, water-absorbing composition.

10. The vegetal-derived, water-absorbing composition according to claim 1, said composition further comprising at least one antioxidant present at a concentration of from about 0.01% to about 5% by weight, based on the total weight of the composition.

11. The vegetal-derived, water-absorbing complex according to claim 10, wherein the at least one antioxidant is selected from the group consisting of: Scutellaria Baicalensis Root Extract; Chamomilla Recutita Extract; Citrus Reticulata Peel Extract; Pueraria Lobata Root Extract; Pueraria Lobata Root Extract; Nordihydroguaiaretic Acid; ascorbic acid and its salts and derivatives; derivatives of ascorbyl esters of fatty acids; tocopherol and esters of tocopherol.

12. A topically-applied personal care product comprising
(i) a vegetal cholesterol derived semi-synthetically from one or a mixture of botanical materials from
(a) a species of the genus Dioscorea selected from the group consisting of D. composita, D. terpinapensis and D. villosa or
(b) Trillium erectum,
(ii) a non-animal, botanically-derived phospholipid comprising at least 20% phosphatidylcholine,
(iii) a botanical oil or ester derivative thereof, said botanical oil or ester derivative thereof being present at a concentration of from 50% to 90% by weight, based on the total weight of the water-absorbing composition,
(iv) at least one sucrose ester at a concentration of from 5% to 10% by weight, based on the total weight of the water-absorbing composition, and
(v) 8% to 15% by weight, based on the total weight of the water-absorbing composition, of fumed silica.

13. The topically-applied personal care product according to claim 12 that is an emulsion.
